(19) United States
(12) Patent Application Publication
(10) Pub. No.: US 2007/0218500 A1
(43) Pub. Date: Sep. 20, 2007

(54) Method and Apparatus for Detection of Live Bacterium Within Test Subject Through Specifically labeling Thereof

(75) Inventors: Tohru Mikoshiba, Tokyo (JP); Tetsuro Sasaki, Osaka (JP); Takaharu Enjoji, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: ENTEST JAPAN, INC.

(21) Appl. No.: 11/587,846

(22) PCT Filed: Mar. 3, 2005
(86) PCT No.: PCT/JP05/03584

§ 371 Date: Mar. 26, 2007

(30) Foreign Application Priority Data
Apr. 28, 2004 (JP) 2004-132476

(51) Int. Cl. G01N 33/53

(52) U.S. Cl. 435/7.1

(57) ABSTRACT

A method and apparatus for detection whereby live bacteria among microbes as an antigen can be detected rapidly in a short period of time through specifically labeling of live bacteria within a test subject antigen and whereby testing assurance can be ensured. The method and apparatus are characterized in that labeled antigen (14) is formed by action, on a test subject antigen such as Escherichia coli, of labeled substance (13) zymolyzable by live bacteria (target bacteria (12)) within the test subject antigen, and the resultant labeled antigen (14) is trapped on an immobilization phase having, immobilized thereon, a specific binding antibody capable of specifically binding to the test subject antigen.

### TECHNICAL FIELD

The present invention relates to a method and an apparatus for detecting a concentration and a number of an antigen in a solution, and in particular, it relates to a method and an apparatus that are capable of detecting a live bacterium in a test subject antigen in a short period of time by specifically labeling the same.

### BACKGROUND ART

In recent years, there are arising problems of damages of food poisoning caused by bacteria, such as salmonella, staphylococcus, botulinum and pathogenic E. coli O-157, and the companies concerned are conducting training sessions and enlightenment programs relating to preventive sanitation against the bacteria, and also trying to prevent proliferation of accidents from occurring in advance through vast business investments.

Bacteria are generally detected by identification and quantitative determination after culture. Specifically, since the detection is associated with a culture operation including pre-culture, enrichment culture and isolation culture, a period of time of about several days is required for obtaining a detection result, and a specialized measurement engineer is required, due to the culture operation. The measurement for a prolonged period of time brings about a severe problem in the case where necessity of a detection test of microorganisms arises in foods, such as fresh foods, which require rapid processing.

Under the circumstances, various test reagents and apparatuses have been developed for detecting pathogenic bacteria of food poisoning easily and rapidly. For example, an immune chromatography method has been widely known, in which a certain bacterium (antigen) is aggregated by using an antibody that is bonded specifically to the certain bacterium through applying immunochemical reaction, and the concentration of the antigen is measured and analyzed. The immune chromatography method will be described in detail below with pathogenic E. coli O-157 having an intrinsic antigen determinant group on the body surface thereof (E. coli having pathogenicity with an antigen determinant group that is bonded specifically to an antibody O-157 exhibited on the bacterium body surface) as an example of the antigen.

The immune chromatography method include an unlabeled immune chromatography method, such as a surface plasmon resonance method, in which the amount of an antigen is measured by utilizing change in physical amount caused by antigen-antibody reaction without a labeling substance, such as an enzyme, bonded to the antibody, and a labeled immune chromatography method, such as a radio immunoassay method, in which an antibody having a labeling substance, such as an enzyme, bonded thereto is used, and the amount of the labeling substance is measured to measure the amount of the antigen, and description herein will be made for the later one, particularly, a sandwich method (sandwich ELISA method), which is a current mainstream owing to the relative easiness in measuring operation thereof.

FIG. 12 is a schematic illustration showing the main process steps of the conventional sandwich method. (a) is an immobilizing step of an antibody (primary antibody), (b) is a trapping step of a target bacterium (antigen), (c) is a staining step with an enzyme-labeled antibody, (d) is an immobilizing step of an antibody (secondary antibody), (e) is an eluting step of a labeled bacterium, and (f) is a detecting step of the eluted labeled bacterium. In FIGS. 12(a) to (f), an immobilizing layer surface 100, a primary antibody 101, a target bacterium 102, a secondary antibody 103, a labeling substance 104, alight source 105 and a detector 106 are shown.

In FIG. 12, a solution containing a primary antibody 101 capable of being bonded specifically to pathogenic E. coli O-157 (target bacterium 102) is placed in a reaction vessel where non-specific adsorption is liable to occur, whereby the primary antibody 101 is adsorbed non-specifically to an immobilizing layer surface 100 of the reaction vessel for immobilization (FIG. 12(a)). A sample solution containing the target bacterium 102 is placed in the reaction vessel to bond the target bacterium 102 specifically to the primary antibody 101 immobilized within the reaction vessel through antigen-antibody reaction (FIG. 12(b)).

Subsequently, a solution containing a secondary antibody 103 labeled with a labeling substance 104 is placed in the reaction vessel, whereby the enzyme-labeled antibody containing the secondary antibody 103 and the labeling substance 104 is bonded specifically to the reaction vessel via the target bacterium 102 through antigen-antibody reaction (FIG. 12(c)). According to the operation, the enzyme-labeled antibody can be immobilized to the reaction vessel in an amount proportional to the target bacterium 102 (FIG. 12(d)). A substrate solution containing a chromogenic substrate is placed in the reaction vessel to color the enzyme-labeled antibody through enzyme reaction.

Finally, the target bacterium 102 bonded with the enzyme-labeled antibody is eluted with a bacteriolytic solution, such as a sodium hydroxide aqueous solution, (FIG. 12(e)), and then light having such a wavelength that is specifically absorbed by the colorant is detected with a detector 106 disposed to face a light source 105, so as to measure the concentration of the antigen (FIG. 12(f)).

As having been described, according to the sandwich method, a bacterium can be appropriately detected rapidly without complex operations and exclusive knowledge.

There has been such a method that a bacterium is detected rapidly by using a test kit capable of being handled easily as compared to the measuring operation of the sandwich method. For example, such a technique has been proposed for detecting rapidly only a live bacterium of E. coli by using a test kit capable of spreading a substrate solution containing a chromogenic substrate component capable of being colored through specific bond with an alkali phosphatase (as disclosed in Patent Document 1).

More specifically, the invention disclosed in Patent Document 1 is a detecting method and a detecting kit containing at least a step of spreading a solution to be detected, in a detecting device containing an immobilized phase containing a specifically bonding component that is capable of bonding specifically to E. coli and is immobilized therein, the immobilized phase being formed on an arbitrary area of a water absorbing substrate, and a step of spreading a substrate solution containing a chromogenic substrate component capable of being colored through specific bond with an alkali phosphatase, on the water absorbing substrate having the solution to be detected having been spread therein.

According to the detecting method and the detecting kit, the spreading speeds of the solution to be detected and the substrate solution can be optimized by appropriately controlling the water absorbing property of the water absorbing substrate, which also speeding up the detection operation. In the case where live bacteria of E. coli are present in the solution to be detected, the chromogenic substrate component is colored by specifically bonding to the alkali phosphatase bonded to the live bacteria trapped in the immobilized phase with the specifically bonding component, so as to provide such an advantage that it can be detected as to whether or not live bacteria of E. coli are present in the solution to be detected. Patent Document 1:

JP-A-2002-165599 (paragraphs [0033] to [0037])

### DISCLOSURE OF THE INVENTION

### Problems to be Solved by the Invention

However, the following problems occur in the sandwich method and the detection method disclosed in Patent Document 1.

In the sandwich method, the two antigen-antibody reaction operations are necessarily effected until detection of the antigen (FIGS. 12(b) and (c)), and a certain period of time is required for each of the antigen-antibody reaction operations. Therefore, there is such a problem that the test cannot be carried out further rapidly. In the sandwich method, furthermore, since an antibody that is bonded specifically to a lipopolysaccharide present on the envelope membrane of E. coli is generally used, it cannot be determined as to whether the E. coli is a live bacterium, or is a killed bacterium or a fragment of bacteria, which brings about such a problem that an acceptable product containing only killed bacteria or fragments of bacteria and causing no damage of food poisoning is rejected upon testing foods.

In the detection method of Patent Document 1, although the detection operation can be speeded up, and a live bacterium of E. coli can be detected, only a small amount of from 0.01 to 0.2 mL of a sample can be handled (as disclosed in Patent Document 1, specification, paragraph [0034]), which brings about such a problem that the certainty of the test cannot be ensured. Specifically, in the case where the amount of the sample to be detected is small, the inclusion probability of live bacteria in E. coli therein is decreased associated with decrease in inclusion probability of E. coli therein, whereby the detection accuracy and the detection sensitivity are necessarily decreased.

In particular, there are many cases even when food poisoning is found in a food factory, the countermeasure is taken after the poisoned food has been sold in retail shops and consumed by consumers. Accordingly, there is a risk of proliferation of food poisoning, and it is demanded to speed up the test.

The invention has been developed in view of the circumstances, and an object thereof is to provide a detecting method and a detecting apparatus capable of detecting a live bacterium among bacteria as an antigen in a short period of time, and capable of ensuring certainty of the test.

### Means for Solving the Problems

In order to solve the problems, the invention is characterized in that a test subject antigen, such as E. coli, is reacted with a labeling substance capable of being enzyme-decomposed with a live bacterium in the test subject antigen to form a labeled antigen, and then the labeled antigen is trapped in an immobilized phase containing, immobilized therein, a specifically bonding antibody capable of bonding specifically to the test subject antigen.

More specifically, the invention provides the following.

(1) A method for detecting a live bacterium in a test subject antigen by specifically labeling the live bacterium through action of the test subject antigen and a labeling substance capable of being enzyme-decomposed with the live bacterium in the test subject antigen, characterized in that the test subject antigen is reacted with the labeling substance to form a labeled antigen capable of being detected optically, and the labeled antigen is trapped in an immobilized phase containing, immobilized therein, a specifically bonding antibody capable of bonding specifically with the test subject antigen.

According to the invention, in a method for detecting a live bacterium in a test subject antigen (including live bacteria and killed bacteria), such as E. coli, through action of the test subject antigen and the labeling substance capable of being enzyme-decomposed with the live bacterium in the test subject antigen, the test subject antigen is reacted with the labeling substance to form a labeled antigen capable of being detected optically, and the labeled antigen is trapped in an immobilized phase containing, immobilized therein, a specifically bonding antibody capable of bonding specifically with the test subject antigen, whereby the target to be trapped includes the labeled antigen (live bacterium) capable of being detected optically and a killed bacterium (fragment of bacteria) that is not labeled and is not capable of being detected optically.

Accordingly, the secondary antibody is not necessary, which has been necessary in the conventional sandwich method, and as a result, the two antigen-antibody reaction operations having been conventionally required can be reduced to only one antigen-antibody reaction operation, which speeds up the test. Furthermore, the antigen capable of being detected optically in the trapped antigen contains only live bacteria as the labeled antigen, whereby live bacteria and killed bacteria can be detected as being distinguished from each other, so as to detect certainly live bacteria, which causes damages of food poisoning.

Moreover, the amount of a test sample handled by the detecting method of the invention is from several tens to several hundreds mL, as compared to the amount of a test sample handled by the conventional detecting method disclosed in Patent Document 1 (0.01 to 0.2 mL), whereby decrease of the inclusion probability of the test subject antigen due to extraction of the sample can be prevented from occurring, and the detection accuracy and the detection sensitivity can be prevented from being decreased to ensure certainty of the test.

(2) The detecting method, characterized in that the labeled antigen having been cultured with a proliferating culture solution is trapped in the immobilized phase.

According to the invention, the labeled antigen having been cultured with a proliferating culture solution is trapped in the immobilized phase, whereby the concentration of the labeled antigen can be increased as compared to the case where a proliferating culture solution is not added, and as a result, the trapping probability of the labeled antigen is increased to improve the detection accuracy and the detection sensitivity.

(3) The detecting method, characterized in that a sample solution containing the labeled antigen is circulated in plural times to trap the circulated labeled antigen in the immobilized phase.

According to the invention, a sample solution containing the labeled antigen is circulated in plural times to trap the circulated labeled antigen in the immobilized phase, whereby the immobilized phase containing the primary antibody immobilized therein can be in contact with the sample solution in plural times, and thus the trapping probability of the labeled antigen is increased to improve the detection accuracy and the detection sensitivity.

(4) The detecting method, characterized in that plural types of the test subject antigens are trapped in plural types of immobilized phases containing, immobilized therein, specifically bonding antibodies capable of bonding specifically with the plural types of the test subject antigens, respectively.

According to the invention, plural types of the test subject antigens are trapped in plural types of immobilized phases containing, immobilized therein, specifically bonding antibodies capable of bonding specifically with the plural types of the test subject antigens, respectively, at one time within one sequence of test operations, whereby the test can be speeded up and improved in efficiency.

(5) A detecting apparatus containing a column capable of containing an immobilized phase containing, immobilized therein, a specifically bonding antibody capable of bonding specifically with a test subject antigen, characterized in that a labeled antigen formed by labeling the test subject antigen is trapped in the column containing the immobilized phase.

According to the invention, in a detection apparatus containing a column (biocolumn) capable of containing an immobilized phase containing, immobilized therein, a specifically bonding antibody capable of bonding specifically with a test subject antigen (including live bacteria and killed bacteria) such as E. coli, a labeled antigen formed by labeling the test subject antigen with a labeling substance capable of labeling only a live bacterium is trapped in the column containing the immobilized phase, whereby only one antigen-antibody reaction operation is necessary for trapping the labeled antigen. Accordingly, the test is speeded up, and live bacteria, which causes damages of food poisoning, can be certainly detected. Furthermore, a large amount of a sample can be handled at one time, whereby decrease of the inclusion probability of the test subject antigen due to extraction of the sample can be prevented from occurring, and the detection accuracy and the detection sensitivity can be prevented from being decreased to ensure certainty of the test.

(6) The detecting apparatus, characterized in that the detecting apparatus further contains a stirring device stirring a liquid, and the labeled antigen is labeled in the stirring device.

According to the invention, the detecting apparatus further contains a stirring device mechanically stirring a liquid (sample solution), and the labeled antigen is labeled in the stirring device, whereby labeling of the test subject antigen is accelerated to produce the labeled antigen efficiently.

(7) The detecting apparatus, characterized in that the column is capable of being used inplural times.

According to the invention, the column is capable of being used in plural times, whereby microorganism detection tests in plural times can be carried out sequentially and efficiently.

(8) A detecting apparatus containing plural columns capable of containing an immobilized phase containing, immobilized therein, a specifically bonding antibody capable of bonding specifically with a test subject antigen, characterized in that a labeled antigen formed by labeling the test subject antigen is trapped in the plural columns containing the immobilized phase.

According to the invention, in a detecting apparatus containing plural (plural types of) columns capable of containing an immobilized phase containing, immobilized therein, a specifically bonding antibody capable of bonding specifically with a test subject antigen, a labeled antigen formed by labeling the test subject antigen is trapped in the plural columns containing the immobilized phase, whereby the test can be speeded up and improved in efficiency.

(9) A biocolumn containing an immobilized phase containing, immobilized therein, a specifically bonding antibody capable of bonding specifically with a test subject antigen.

According to the invention, such a biocolumn is provided that contains an immobilized phase containing, immobilized therein, a specifically bonding antibody capable of bonding specifically with a test subject antigen, whereby the test can be speeded up and improved in efficiency. The biocolumn can be stored in a stable state for a prolonged period of time by using such storing means as a freeze-drying method.

(10) A method for stirring an immobilized phase containing, immobilized therein, a specifically bonding antibody capable of bonding specifically with a test subject antigen, by utilizing a pressure fluctuation in a biocolumn containing the immobilized phase.

According to the invention, a rapid pressure fluctuation is caused in the biocolumn to increase the flow rate of a test solution flowing inside the biocolumn, whereby the immobilized phase is stirred in the biocolumn. Accordingly, the immobilized phase can be sufficiently made in contact with a sample solution (test solution) to maintain the detection accuracy and the detection sensitivity at high levels.

### ADVANTAGE OF THE INVENTION

As having been described, according to the invention, the two antigen-antibody reaction operations having been conventionally required can be reduced to only one antigen-antibody reaction operation, which speeds up the test, and only the labeled antigen (live bacterium) is optically detected to distinguish live bacteria and killed bacteria from each other. Furthermore, a large amount of a sample can be handled to ensure certainty of the test.

### BEST MODE FOR CARRYING OUT THE INVENTION

The best mode for carrying out the invention will be described below with reference to the drawings.

[Detecting Apparatus]

FIG. 1 is an appearance view showing a detecting apparatus 1 according to an embodiment of the invention.

In FIG. 1, the detecting apparatus 1 according to the embodiment of the invention has a biocolumn 2 containing an immobilized phase trapping a target bacterium, on a side wall of a control box in a box form having a pump and a valve therein. An M-Cell 3 containing a bacteriolytic solution eluting the target bacterium after trapping is provided adjacent to the biocolumn 2, and on an upper surface of the detecting apparatus 1, for example, five bottles B1 to B5 (the number thereof is not limited) are provided.

FIG. 2 is an enlarged illustration of the biocolumn 2 provided in the detecting apparatus 1 of according to the embodiment of the invention. In FIG. 2, the biocolumn 2 is produced by filling glass beads capable of trapping an antigen through antigen-antibody reaction, in a glass tube for a biocolumn.

More specifically, glass beads are pre-treated with a sodium hydroxide aqueous solution or hydrochloric acid and then dried over night. The glass beads are subjected to a sintering treatment and a silylation treatment with a silylating agent, and they are rinsed and dried at room temperature to produce silylated glass beads.

Subsequently, about 0.5 g of the silylated glass beads are filled in a glass tube for a biocolumn. They are immersed in a glutaraldehyde solution containing glutaraldehyde as a coupling agent for several tens minutes, and after rinsing with a phosphate buffer solution, a primary antibody is immobilized therein through non-specific adsorption. In the immobilization treatment of the primary antibody, the glass tube for a biocolumn is appropriately rinsed to remove the primary antibody unreacted.

After completing the immobilization treatment of the primary antibody, a blocking solution containing bovine albumin serum as a blocking agent is then charged thereto to non-specifically adsorbing the blocking agent to the non-specific adsorbing surface remaining on the surface of the silylated glass beads, whereby subsequent non-specific adsorption of other organic substances is prevented from occurring.

Finally, the interior of the glass tube for a biocolumn is rinsed with a phosphate buffer solution or the like in plural times to remove the blocking agent unreacted, whereby the biocolumn 2 is completed. The biocolumn 2 can be stored in a stable state for a prolonged period of time by using such storing means as a freeze-drying method.

The production of the biocolumn 2 using glass beads has been described in the embodiment of the invention, but the invention is not limited thereto, and for example, flat glass, which is relatively easily handled, may be used for ensuring the conditions of the silylation treatment and the coupling treatment. The spherical glass beads are used in the embodiment of the invention, but the glass beads are one example of a carrier for immobilizing an antibody (i.e., a matrix for immobilizing an antibody), and a carrier in any shape may be used, as far as it has such a surface area that is capable of immobilizing an antibody and has such a shape that is capable of being sufficiently in contact with an antibody and a test solution in the state where the carrier is filled in the column. The production process is substantially the same as the production process of the biocolumn 2 using the glass beads, and the explanation is omitted. The invention can be applied irrespective of the material of beads, the conditions on the silylation treatment, and the method of immobilizing the primary antibody, as far as the primary antibody can be immobilized on the immobilizing layer surface.

[Detecting Process]

FIG. 3 is a flow path schematic diagram upon detecting microorganisms by using the detecting apparatus 1 shown in FIG. 1. FIG. 4 is a flow chart outlining the detecting process in the flow path schematic diagram shown in FIG. 3.

In FIG. 3, the bottle B1 contains a test solution (for example, 100 mL) containing a test subject antigen containing a mixture of a live bacterium having been labeled through hydrolysis (labeled antigen) and a killed bacterium having not been subjected to hydrolysis, by adding 6-carboxyl fluorescein diacetate diluted (CFDA diluted) solution as a staining agent (solution containing a labeling substance), the bottles B5 and B6 contain a phosphate buffer solution as a column rinsing solution, and M-Cell 3 contains an alkali aqueous solution eluting the target bacterium trapped in the biocolumn 2. CFDA used in the embodiment may be replaced by such an agent that is capable of staining a live bacterium, which can be detected through fluorescence or the like.

In FIG. 4, an immobilizing step is first effected (Step S1). More specifically, in FIG. 3, the test solution added with the CFDA diluted solution charged in the bottle B1 flows from the bottle B1, a valve V1, a valve V2, a pump P1, a valve V3, the biocolumn 2, a valve V4, a valve V5, a valve V6 to a bottle B2 in this order by operating the pump P1. The period of time required is about 15 minutes. The test solution stored in the bottle B2 flows from the bottle B2, the valve V1, the valve V2, the pump P1, the valve V3, the biocolumn 2, the valve V4, the valve V5, the valve V6 to a bottle B3 by switching the valve V1. The period of time required is also about 15 minutes. According to the immobilizing step S1, the test subject antigen in the test solution is bonded specifically to the primary antibody immobilized to the biocolumn 2 (glass beads) through antigen-antibody reaction. It is possible that a proliferating culture solution is added to the bottle B1, and the labeled antigen having been cultured with the proliferating culture solution is trapped. According to the operation, the concentration of the labeled antigen can be increased to improve the trapping probability of the labeled antigen.

In order to maintain the detection accuracy and the detection sensitivity at high levels, it is necessary that the immobilized phase (glass beads) having the specifically bonding antibody immobilized therein is made sufficiently in contact with the circulating sample solution (test solution). In the embodiment, the glass beads (immobilized phase) in the biocolumn 2 are efficiently stirred by utilizing an electromagnetic pinch valve PV (as shown in FIG. 3). This will be described more specifically with reference to FIG. 5. FIG. 5 is an explanatory view showing the state where the immobilized phase is efficiently stirred.

In FIG. 5, the pinch valve PV, which is provided between the biocolumn 2 and the valve V3, is changed from the open state (ON state) to the closed state (OFF state) (left figure to center figure in FIG. 5) the test solution stops flowing from the pinch valve PV to the biocolumn 2, and the pressure in the pipe between the pinch valve PV and the valve V3 is increased. After lapsing a prescribed period of time, the pinch valve PV is changed from the closed state (OFF state) to the open state (ON state) (center figure to right figure in FIG. 5), the test solution again starts flowing from the pinch valve PV to the biocolumn 2.

At this time, a rapid pressure fluctuation occurs in the biocolumn 2 due to the closed state (OFF state) of the pinch valve PV for the prescribed period of time, whereby the flow rate of the test solution flowing in the biocolumn 2 is increased. As a result, the glass beads (immobilized phase) are stirred in the biocolumn 2 (as shown in right figure in FIG. 5).

As having been described, in the embodiment, the pinch valve PV is opened and closed at a prescribed timing upon circulating the test solution through the biocolumn 2, whereby the immobilized phase (glass beads) is periodically and efficiently stirred. According to the constitution, the immobilized phase (glass beads) can be made sufficiently in contact with the test solution.

In the embodiment, the electromagnetic pinch valve PV is used, but the invention is not limited thereto, and for example, a manumotive or electromotive pinch valve may be used. Furthermore, any means that is capable of stirring the immobilized phase in the biocolumn 2 appropriately may be used, and the invention is not particularly limited to a pinch valve.

A rinsing step is then effected (Step S2). More specifically, in FIG. 3, the valve V2 is switched, and a phosphate buffer solution stored in a bottle B5 flows from the bottle B5, the valve V2, the pump P1, the valve V3, the biocolumn 2, the valve V4, the valve V5 to a bottle B4 in this order. Thereafter, the pump Pi is turned off. The period of time required is about 15 minutes. According to the rinsing step of Step S2, the phosphate buffer solution flows through the biocolumn 2 to rinse away the primary antibody unreacted and the like to effect, as a result, condensation of the test subject antigen.

An eluting step is then effected (Step S3). More specifically, in FIG. 3, the valve V3 and the valve V4 are switched, and the M-Cell 3 containing a bacteriolytic solution eluting the test subject antigen and a pump P2 are operated, whereby the test subject antigen trapped in the biocolumn 2 is eluted. A labeled live bacterium (labeled antigen) in the test subject antigen is optically detected (spectral measurement) with a fluorescence spectrophotometer equipped with a flow cell (at the right lower part in FIG. 3). According to the eluting step of Step S3, only live bacteria, which causes damages of food poisoning, are detected. The microorganism test is tentatively completed by the sequence of steps of from Step S1 to Step S3.

In the case where the microorganism tests are carried out sequentially by charging the solutions for experiments in several times in the bottles B4 and B6, the rinsing step is additionally effected (Step S4). More specifically, in FIG. 3, the pump P2 is operated, and the valve V4 and a valve V7 are switched, whereby the biocolumn 2 is rinsed with the phosphate buffer solution stored in the bottle BG.

As having been described, it is understood that only a live bacterium can be detected among microorganisms as an antigen according to the sequence of detecting process steps of the Step S1 to Step S3 (Step S4) shown in FIG. 4. The amount of a test solution capable of being detected by the detecting apparatus 1 according to the embodiment of the invention is from several tens to several hundreds mL, which is different from the amount of a test solution handled by the detecting kit (about from 0.01 to 0.2 mL), whereby decrease of the inclusion probability of E. coli due to sampling can be prevented from occurring, and the detection accuracy and the detection sensitivity can be improved. The agents and the methods used in the steps of rinsing, eluting and rinsing may be changed unless the gist of the invention is deviated.

According to the sequence of detecting process steps of the Step S1 to Step S3 (Step S4) shown in FIG. 4, furthermore, the detection test of microorganisms can be completed in a shorter period of time than the conventional sandwich method. The speeding up of the detection test by using the detecting apparatus 1 will be described in detail below with reference to the schematic illustration shown in FIG. 6.

[Schematic Illustration]

FIG. 6 is a schematic illustration showing the main process steps of the detecting method according to the embodiment of the invention. (a) is an immobilizing step of an antibody (primary antibody), (b) is stirring step of a sample solution containing the test subject antigen, and a fluorescent agent, (c) is a trapping step of the test subject antigen containing a labeled antigen, (d) is an immobilizing step of the test subject antigen, (e) is an eluting step of the test subject antigen, and (f) is a detecting step of only the labeled antigen in the eluted test subject antigen. In FIGS. 6(a) to (f), an immobilizing layer surface 10, a primary antibody 11, a target bacterium (live bacterium) 12, a labeling substance 13, a labeled antigen 14, a light source 15 and a detector 16 are shown.

In FIG. 6, the primary antibody 11 is adsorbed non-specifically to the immobilizing layer surface 10 of the glass beads filled in the glass tube for a biocolumn (FIG. 6(a)). The details of this step have been described for the production process of the biocolumn 2.

A fluorescent agent is then added to a sample solution containing the test subject antigen to make the target live bacterium 12 luminescent (FIG. 6(b)). More specifically, upon adding a CFDA diluted solution to the sample solution, a live bacterium in the test subject antigen absorbs CFDA (labeling substance 13) as an intracellular pH indicator and produces fluorescence through hydrolysis. In other words, CFDA exerts a function as a live bacterium staining agent. After adding the CFDA diluted solution to the sample solution, the hydrolysis with the live bacterium may be-accelerated by stirring with a stirring device. According to the operation, CFDA can be absorbed by the live bacterium certainly within a shorter period of time, whereby the detection test can be speeded up.

The sample solution having the test subject antigen (containing the labeled antigen 14) present therein is then made in contact with the immobilizing layer surface 10 in the biocolumn 2, whereby the test subject antigen is trapped through antigen-antibody reaction with the primary antibody 11 (FIG. 6(c)). After trapping the test subject antigen, a rinsing solution, such as a phosphate buffer solution, is made flow into the biocolumn 2 to remove impurities and the primary antibody unreacted, whereby condensation (concentration) of the test subject antigen and the immobilization of the test subject antigen are effected (FIG. 6(d)). It is preferred that the stirring step shown in FIG. 6(b), the trapping step shown in FIG. 6(c) and the immobilizing step shown in FIG. 6(d) are repeatedly effected in plural times. According to the operation, the number of the test subject antigen unreacted can be decreased to improve the detection accuracy and the detection sensitivity.

The test subject antigen immobilized with the primary antibody 11 and containing the labeled antigen 14 is then subjected to bacteriolysis and elution with an alkali solution (FIG. 6(e)). At this time, the volume inside the circulation path and the volume of the flow cell are decreased to decrease the amount of the alkali solution required for the bacteriolysis and elution, whereby the concentration of bacteria in the resulting bacteriolytically eluted solution can be increased to improve the detection sensitivity. In the embodiment of the invention, a high concentration alkali solution is used, but for example, an acidic buffer solution or a surfactant may be used in combination to attain bacteriolysis and elution of the test subject antigen more rapidly and certainly.

Finally, the labeled antigen 14 is optically detected with the detector 16 disposed to face the light source 15 (FIG. 6(f)). More specifically, the labeled antigen 14 containing the labeling substance 13 produces fluorescence through excitation with an ultraviolet ray emitted from the light source 15, and the fluorescence is detected with the detector 16 equipped with a condensing lens to take out an electric signal (chromatographic signal). The electric signal is measured and analyzed to detect optically the labeled antigen 14 (target bacterium 12). In the embodiment of the invention, a fluorescence spectrophotometer is used, but the embodiment for detection is not limited, and for example, such a detector as a particle counter may be used.

As having been described, according to the sequence of process steps shown in FIGS. 6(a) to 6(f), the detection test of microorganisms can be carried out in a shorter period of time than the conventional sandwich method. Specifically, in the conventional sandwich method, the two antigen-antibody reaction operations are necessarily effected until detection of an antigen (as shown in FIGS. 12(b) and (c)), but according to the invention, only one antigen-antibody reaction operation of the labeled antigen and the primary antibody 11 may be effected to reduce the period of time required for the test, whereby the test can be speeded up.

[Modified Example]

FIG. 7 is an appearance view showing a detecting apparatus according to another embodiment of the invention. Characteristic features thereof include provision of two biocolumns 65 and 66 capable of trapping specific target bacteria. In FIG. 7, two biocolumns 65 and 66 are provided, but the invention is not limited thereto, and for example, three or more biocolumns may be provided. In the case where plural biocolumns are provided, plural types of target bacteria can be simultaneously detected.

In FIG. 7, in the detecting apparatus according to another embodiment of the invention, the devices, pumps and bottles are placed in a constant-temperature chamber (rectangle frame in the figure) at 35±1° C., and the devices and pumps are optimally controlled with a flow path controlling sequencer 69. Inside the constant-temperature chamber, a sample supplying chamber (sample hopper) 61 supplying a test sample containing a target bacterium, a stirring device (magnetic stirrer) 62 stirring the sample, a filter 63 removing impurities, a flow path switching valve 64 switching the flow path appropriately, biocolumns 65 and 66 filled with glass fine particles having an antibody for the target bacterium immobilized on the surface thereof, a circulation pump 67 circulating the sample, and a high sensitivity fluorescence detector 68 detecting the target bacterium optically are placed, and a bottle B11 containing a rinsing solution, a bottle B12 containing an immobilizing solution, and a bottle B13 containing a bacteriolytic elution solution for a stained bacterium trapped are provided. The detecting process using the detecting apparatus shown in FIG. 7 will be outlined below.

A prescribed amount of a sample is subjected to stomaching by an ordinary method, and a sample solution (50 to 100 mL) is placed in the sample supplying chamber 61. Under stirring with the stirring device 62, CFDA as a fluorescent staining agent is added thereto to stain a live bacterium. After stirring for about 10 minutes, the sample solution is filtered with the filter 63 to remove impurities and introduced into the sample flow path (biocolumn 65). The flow path switching valve 64 is then switched to the filter rinsing system to rinse the filter 63.

The sample solution passing through the filter unit (filter 63) is circulated in the entire flow path and rinsing path through the biocolumns 65 and 66, whereby the sample solution passes through the biocolumns 65 and 66 in several times. Thereafter, the stained bacterium (labeled antigen) trapped by the immobilized antibody is eluted to a high concentration by circulating a small amount of the bacteriolytic elution solution supplied from the bottle B13 to the recycle flow path in the biocolumns 65 and 66 in several times. The sample solution having been eluted is introduced to the high sensitivity fluorescence detector 68 through the switching valve 64 under the biocolumns 65 and 66 to draw a chromatogram as an electric signal.

More specifically, the sample solution having been eluted is introduced to a flow cell of the high sensitivity fluorescence detector 68, and the stained bacterium in the sample solution produces fluorescence through excitation with an ultraviolet ray emitted from the light source. The fluorescence is received through a condensing lens, and an optical signal is converted to an electric signal to draw a chromatogram.

Finally, after completing the bacteriolysis and elution, the biocolumns 65 and 66 filled with the glass fine particles having the antibody for the target bacterium immobilized thereon are refreshed by rinsing with the rinsing solution in the bottle B11.

As having been outlined, according to the detecting apparatus shown in FIG. 7, two biocolumns 65 and 66 are provided to trap two types of target bacteria simultaneously in one sequence of detection, whereby the test can be speeded up and improved in efficiency.

Plural types of target bacteria can be simultaneously detected by providing plural biocolumns having different antibodies in series. Plural test solutions for a specific target bacterium can be simultaneously detected by providing biocolumns having the same antibody in parallel. Both these configurations may be used in combination.

[Culturing Step]

In the detecting method according to the embodiment of the invention, a target bacterium can be basically detected sufficiently without a culturing step. However, a target bacterium may be cultured depending on necessity, whereby a more definitive test result can be obtained. The culturing step may be effected, for example, by culturing the bacterium before the immobilizing step with a heater provided in the detecting apparatus, or by culturing the bacterium by providing the entire detecting apparatus for the detecting process is maintained at a prescribed temperature.

### Example 1

FIG. 8 is a diagram showing measurement results in a performance test of the biocolumn 2 in the flow path shown in FIG. 3. More specifically, it shows the CFDA fluorescent intensity with respect to the charged amount (CFU per 100 mL) of E. coli. According to the table shown in FIG. 8, it is understood that there is a high correlativity between the charged amount of E. coli and the CFDA fluorescent intensity in the bacteriolytically eluted solution. According to FIG. 9 showing a graph obtained by plotting the data in the table shown in FIG. 8 in a two-dimensional field, the CFDA fluorescent intensity is increased with increase of the charged amount of E. coli, and thus it is understood that the target bacterium has been appropriately detected.

In the table shown in FIG. 8, a relatively strong fluorescent spectrum (1.2050 in average) is obtained for the test solution having the minimum charged amount of E. coli of 10 CFU/mL, and therefore, it is considered that a target bacterium can be suitably detected for a test solution of about 5 CFU/mL. Furthermore, it is also considered that a target bacterium can be suitably detected for a test solution of about from 1 to 5 CFU/mL by decreasing the volume inside the circulation path and the volume of the microflow cell to decrease the necessary amount of the bacteriolytic elution solution.

FIG. 10 is a diagram showing measurement results in a performance test of the biocolumn 2 in the flow path shown in FIG. 3. FIG. 10(a) shows the CFDA fluorescent intensity of killed bacteria having been stained. According to the table shown in FIG. 10(a), it is understood that the CFDA fluorescent intensity in the bacteriolytic elution solution is significantly weak even when killed bacteria are charged as the test subject antigen. In other words, even when killed bacteria, which cause no damage of food poisoning, are present in a test solution, only live bacteria can be detected with high accuracy without interference of the killed bacteria, whereby the problem of rejecting an acceptable product containing only killed bacteria can be solved.

FIG. 10(b) shows the CFDA fluorescent intensity to several kinds of bacteria (coliform group bacteria: C. freundii, and Enterobacteriaceae: S. marcescens) other than E. coli with respect to the charged amount (CFU per 100 mL). According to the table shown in FIG. 10, it is understood that the CFDA fluorescent intensity of bacteria other than E. coli is weak. In other words, as similar to the case of killed bacteria, even when bacteria other than the target bacterium are present in a test solution, the influence thereof can be relatively low.

FIG. 11 is a table showing measurement results in a performance test of the biocolumn 2 repeatedly used in the flow path shown in FIG. 3. More specifically, it shows the CFDA fluorescent intensity with respect to the accumulated number of use of the biocolumn 2. According to FIG. 11, it is understood that when the biocolumn 2 is used twice, the CFDA intensity in the bacteriolytic elusion solution is decreased by about 98% in the second use. It is considered that this is because a high concentration alkali solution having bacteriolytic function is used for elution of the target bacterium, and the antibody, which is protein, is also damaged thereon along with the bacterium. Accordingly, the biocolumn 2 can be repeatedly used by using, for example, such a bacteriolytic elusion solution that causes no damage on the antibody.

An evaluation test where the species and amounts of the test materials are changed will be outlined below.

100 mL of a bacterium solution, the number of bacteria of which has been estimated by the MPN method or the like, is placed in a sample supplying bottle of the test apparatus, to which 1 mL of a live bacterium staining solution containing CFDA is added, and the entire amount thereof is circulated twice in the biocolumn at a flow rate of 10 mL/min, followed by draining away. After applying the entire amount of the test solution to the biocolumn, the interior of the biocolumn is rinsed by flowing a suitable amount of a biocolumn rinsing solution in the biocolumn, and the flow path is switched to drain away the entire biocolumn rinsing solution remaining in the biocolumn.

The target bacteria having been stained for live bacteria therein and trapped by the biocolumn are bacteriolytically eluted by using a bacteriolytic elution solution in a total amount of 10 mL, and introduced into the flow cell of the fluorescence spectrophotometer to measure the fluorescent intensity. After completing the measurement, the entire flow path is rinsed with a sterilized diluted phosphate buffer solution.

In the evaluation test having been outlined, the period of time requires is about 1 hour. It is understood in the evaluation test that the capability of detection can be sufficiently exerted when about 30 CFU or more of live bacteria are present in the sample. It is also understood that the influence of bacteria other than E. coli (coliform group bacteria and Enterobacteriaceae) and the influence of killed bacteria are considerably small to provide no problem on practical use. E. coli has been exemplified as a target of the invention, but any one capable of being trapped through antigen-antibody reaction can be used as a target, and for example, fungi can be used as a target.

### INDUSTRIAL APPLICABILITY

The detecting method and the detecting apparatus of the invention as useful in such a point that as a test subject antigen, a labeled antigen having been reacted with a labeling substance capable of being decomposed through enzyme reaction with live bacteria in the test subject antigen is detected, whereby only a live bacterium in the test solution can be a target bacterium, so as to ensure speeding up and certainty of the test.

### BRIEF DESCRIPTION OF THE DRAWINGS

[FIG. 1]

FIG. 1 is an appearance view showing a detecting apparatus 1 according to an embodiment of the invention.

[FIG. 2]

FIG. 2 is an enlarged illustration of a biocolumn provided in the detecting apparatus according to the embodiment of the invention.

[FIG. 3]

FIG. 3 is a flow path schematic diagram upon detecting microorganisms by using the detecting apparatus shown in FIG. 1.

[FIG. 4]

FIG. 4 is a flow chart outlining the detecting process in the flow path schematic diagram shown in FIG. 3.

[FIG. 5]

FIG. 5 is an explanatory view showing the state where the immobilized phase is efficiently stirred.

[FIG. 6]

FIG. 6 is a schematic illustration showing the main process steps of the detecting method according to an embodiment of the invention.

[FIG. 7]

FIG. 7 is an appearance view showing a detecting apparatus according to another embodiment of the invention.

[FIG. 8]

FIG. 8 is a diagram showing measurement results in a performance test of a biocolumn in the flow path shown in FIG. 3.

[FIG. 9]

FIG. 9 is a graph obtained by plotting the data in the table shown in FIG. 8 in a two-dimensional field.

[FIG. 10]

FIG. 10 is a diagram showing measurement results in a performance test of a biocolumn in the flow path shown in FIG. 3.

[FIG. 11]

FIG. 11 is a table showing measurement results in a performance test of a biocolumn repeatedly used in the flow path shown in FIG. 3.

[FIG. 12]

FIG. 12 is a schematic illustration showing main process steps of a conventional sandwich method.

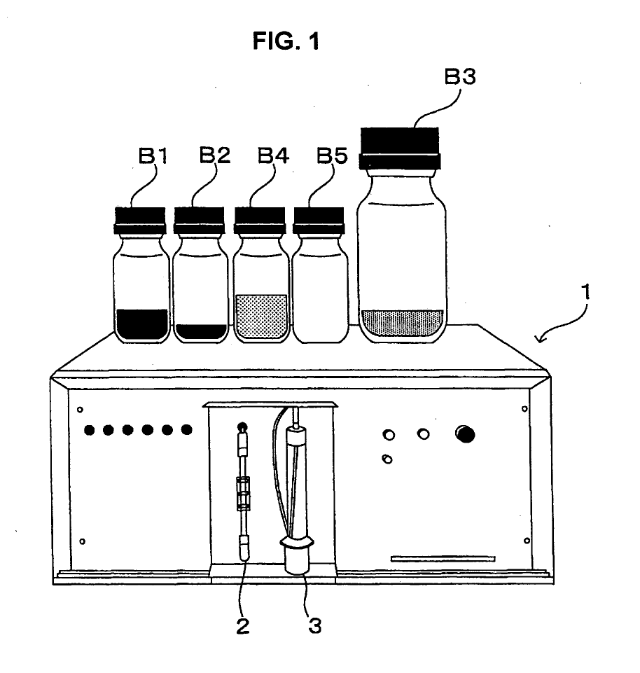

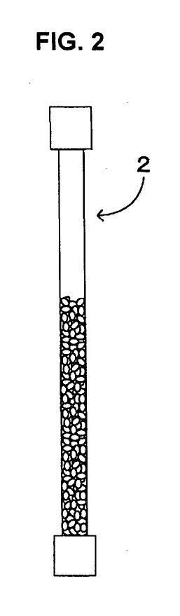

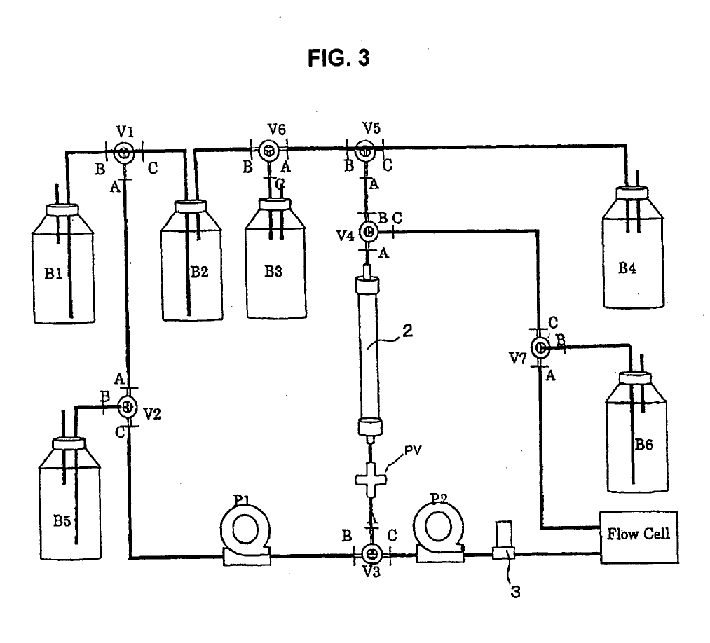

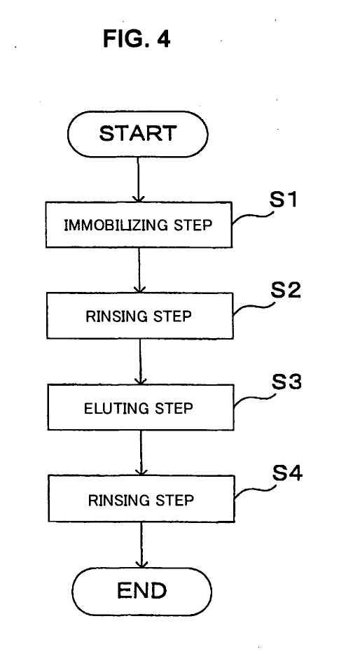

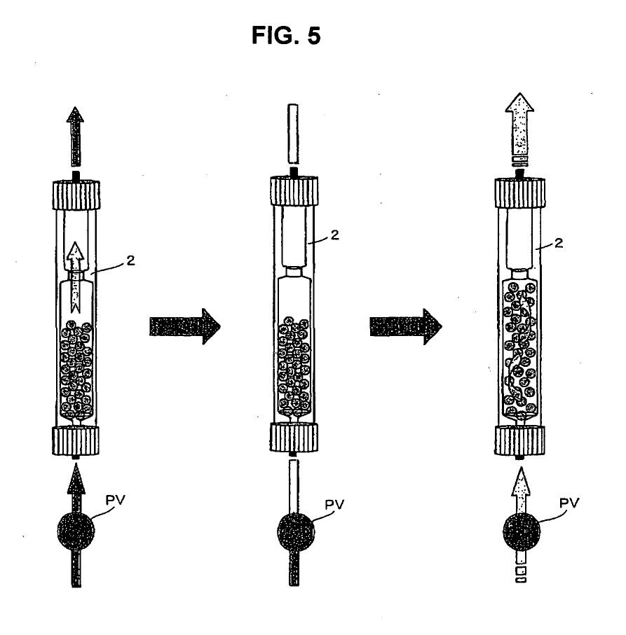

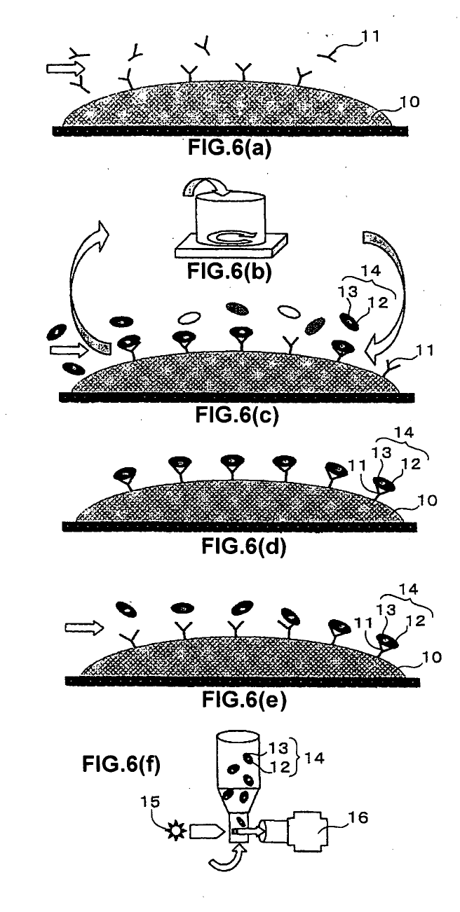

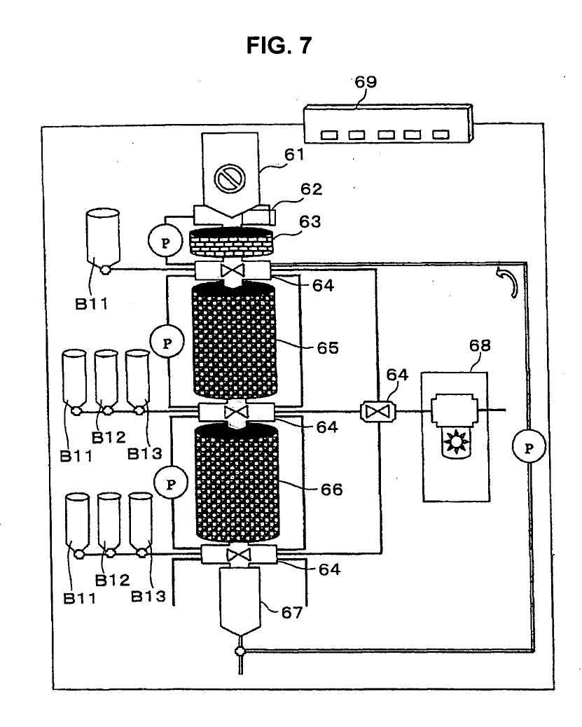

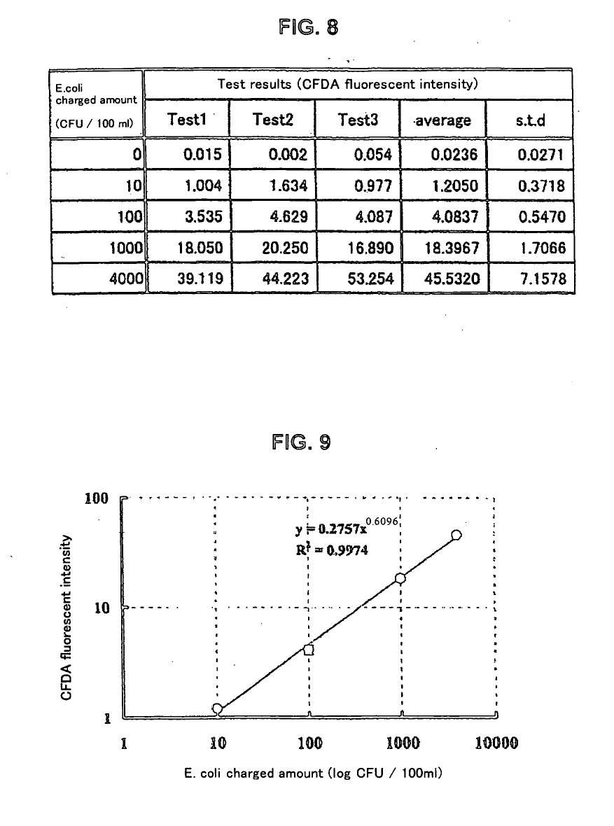

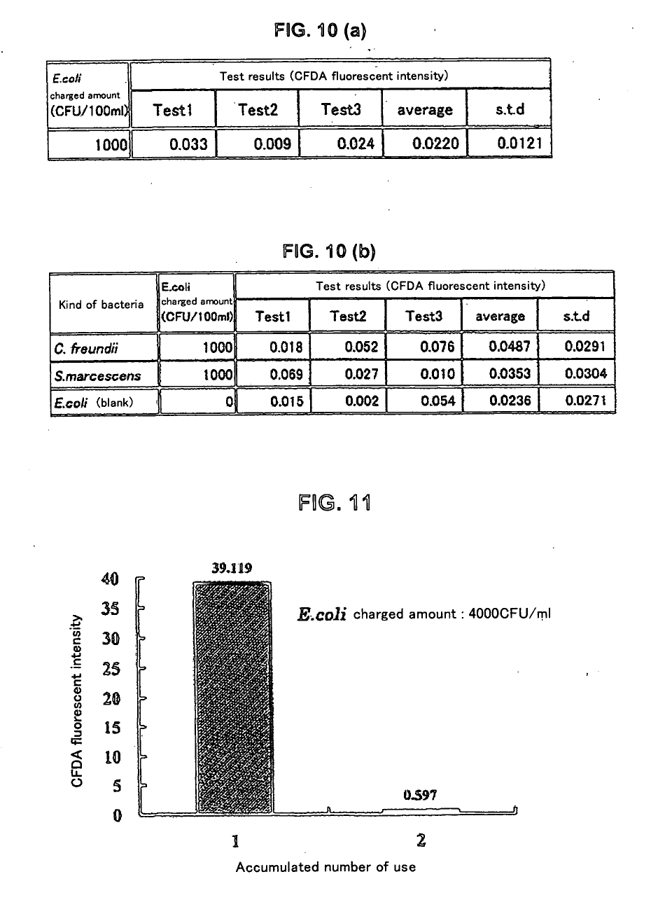

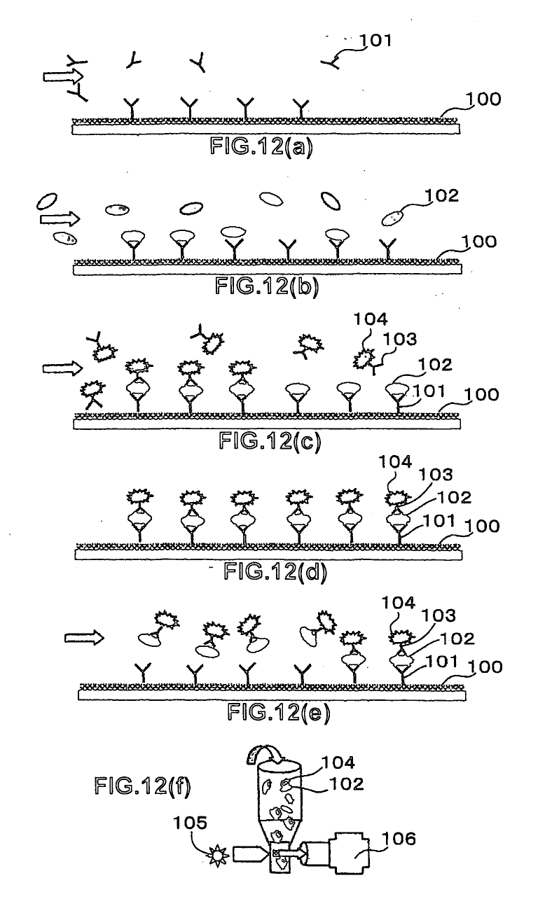

### DESCRIPTION OF SYMBOLS

1 detecting apparatus
2 biocolumn
3 M-Cell
10 immobilizing layer surface
11 primary antibody
12 target bacterium (live bacterium)
13 labeling substance
14 labeled antigen
15 light source
16 detector

1. A method for detecting a live bacterium in a test subject antigen by specifically labeling the live bacterium through action of the test subject antigen and a labeling substance capable of being enzyme-decomposed with the live bacterium in the test subject antigen, characterized in that
the test subject antigen is reacted with the labeling substance to form a labeled antigen capable of being detected optically, and
the labeled antigen is trapped in an immobilized phase containing, immobilized therein, a specifically bonding antibody capable of bonding specifically with the test subject antigen.

2. The detecting method according to claim 1, characterized in that the labeled antigen having been cultured with a proliferating culture solution is trapped in the immobilized phase.

3. The detecting method according to claim 1, characterized in that a sample solution containing the labeled antigen is circulated in plural times to trap the circulated labeled antigen in the immobilized phase.

4. The detecting method according to claim 1, characterized in that plural types of the test subject antigens are trapped in plural types of immobilized phases containing, immobilized therein, specifically bonding antibodies capable of bonding specifically with the plural types of the test subject antigens, respectively.

5. A detecting apparatus comprising a column capable of containing an immobilized phase containing, immobilized therein, a specifically bonding antibody capable of bonding specifically with a test subject antigen, characterized in that
a labeled antigen formed by labeling the test subject antigen is trapped in the column containing the immobilized phase.

6. The detecting apparatus according to claim 5, characterized in that the detecting apparatus further comprises a stirring device stirring a liquid, and
the labeled antigen is labeled in the stirring device.

7. The detecting apparatus according to claim 5, characterized in that the column is capable of being used in plural times.

8. A detecting apparatus comprising plural columns capable of containing an immobilized phase containing, immobilized therein, a specifically bonding antibody capable of bonding specifically with a test subject antigen, characterized in that
a labeled antigen formed by labeling the test subject antigen is trapped in the plural columns containing the immobilized phase.

9. A biocolumn comprising an immobilized phase containing, immobilized therein, a specifically bonding antibody capable of bonding specifically with a test subject antigen.

10. A method for stirring an immobilized phase containing, immobilized therein, a specifically bonding antibody capable of bonding specifically with a test subject antigen, by utilizing a pressure fluctuation in a biocolumn containing the immobilized phase.
